(19) United States
(12) Patent Application Publication
(10) Pub. No.: US 2016/0199255 A1
(43) Pub. Date: Jul. 14, 2016

(54) Acupressure Device and Method of Use

(71) Applicant: Ross G. Drago, Berkeley, CA (US)

(72) Inventor: Ross G. Drago, Berkeley, CA (US)

(21) Appl. No.: 14/592,018

(22) Filed: Jan. 8, 2015

(51) Int. Cl. A61H 39/04; A61B 18/18

(52) U.S. Cl. CPC A61H 39/04; A61B 18/18

(57) ABSTRACT

An apparatus and method for administering acupressure are disclosed. In one embodiment, the apparatus comprises an electrically-conductive elongated member to be applied to one or more acupressure points of a person, and an electrically-conductive ground plug, coupled via an electrically-conductive wire to the elongated member, to be inserted into a ground receptacle of an alternating-current outlet during application of the elongated member to the one or more acupressure points. The ground plug and wire enable charge drainage from the person during application of the elongated member to the one or more acupressure points.

### FIELD OF THE INVENTION

moon The present invention relates to acupressure, and, more particularly, an apparatus and method for administering acupressure.

### BACKGROUND OF THE INVENTION

Acupressure is an alternative-medicine technique based on the concept of life energy flowing through “meridians” in the body. In acupressure treatment, physical pressure is applied to one or more acupressure points of a person's body with the aim of balancing the meridians and clearing any blockages. Acupressure has been shown to improve health by increasing circulation, reducing pain, and relieving muscle tension.

### SUMMARY OF THE INVENTION

An apparatus and method for administering acupressure are disclosed. In one embodiment, an apparatus comprises an electrically-conductive elongated member that a first person (referred to hereinafter as the “therapist”) uses to apply physical pressure to an acupressure point of a second person (referred to hereinafter as the “patient”), and an electrically-conductive wire coupled to the electrically-conductive elongated member. In one embodiment, the wire is to be inserted into a ground receptacle of an alternating-current outlet, thereby enabling excess charge (e.g., of the related meridian, etc.) to be drained from the patient. Studies have demonstrated that draining excess charge from one's body can provide numerous health benefits, including improvements in blood viscosity, heart rate variability, cortisol dynamics, sleep, and autonomic nervous system balance, as well as reduced inflammation and stress. Embodiments of the present disclosure are thus capable of providing the health benefits of acupressure, as well as further health benefits associated with draining excess charge.

Advantageously, embodiments of the present disclosure can benefit the therapist administering acupressure, as well as benefitting the patient. In particular, by directing the patient's excess charge into the ground, rather than into the therapist's own body, embodiments of the present disclosure can protect the therapist from developing the very health problems (e.g., inflammation, etc.) being relieved in the patient. Moreover, embodiments of the present disclosure can be used for self-administration, thereby providing the same health benefits as administration by a therapist.

### BRIEF DESCRIPTION OF THE DRAWINGS

FIG. 1 depicts a first embodiment of an apparatus for administering acupressure.

FIG. 2 depicts an illustrative coupling of wire 104 to endcap 102 and elongated member 101, as shown in FIG. 1, in accordance with one embodiment of the present disclosure.

FIG. 3 depicts an illustrative use of apparatus 100 in accordance with one embodiment of the present disclosure.

### DETAILED DESCRIPTION

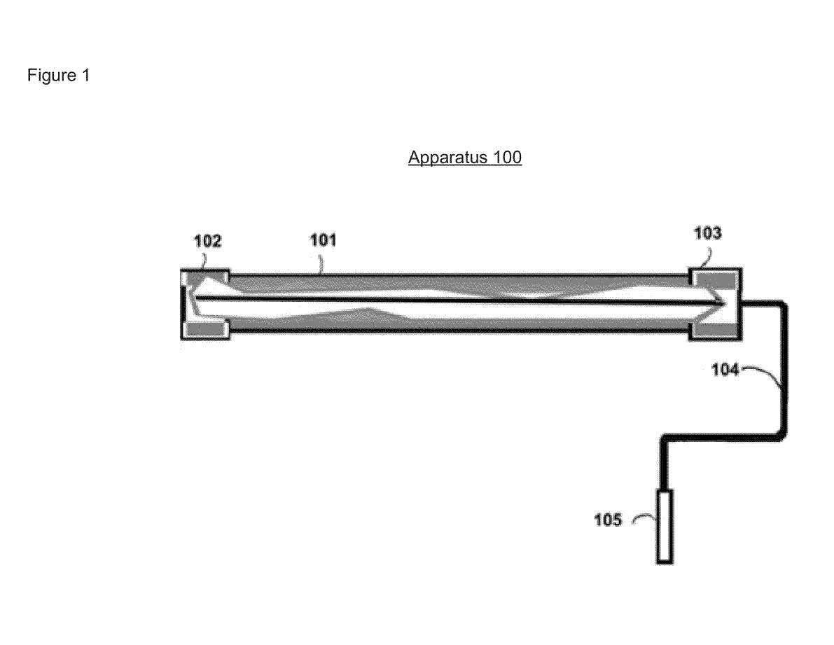

FIG. 1 depicts an apparatus 100 for administering acupressure, in accordance with one embodiment. As shown in FIG. 1, apparatus 100 comprises elongated member 101, endcap 102, endcap 103, wire 104, and grounding plug 105 interconnected as shown.

In one embodiment, elongated member 101 is an electrically-conductive tube that is open at both ends. In some alternative embodiments, elongated member 101 might be something other than a tube (e.g., a solid bar [i.e., a bar that has no voids], etc.); similarly, in some other embodiments, one or both ends of elongated member 101 might be sealed, rather than open. It will be clear to those skilled in the art, after reading this disclosure, how to make and use such alternative embodiments of elongated member 101.

In one embodiment, elongated member 101 comprises copper. As will be appreciated by those skilled in the art, in some other embodiments elongated member 101 might comprise some other electrically-conductive material (e.g., aluminum, steel, etc.), either instead of copper, or in addition to copper.

In one embodiment, elongated member 101 is cylindrical. As will be appreciated by those skilled in the art, in some other embodiments elongated member 101 might have a non-circular cross section (e.g., elliptical, rectangular, hexagonal, etc.), or might have a non-uniform cross-section (e.g., a cone-shaped elongated member 101, etc.).

In one embodiment, elongated member 101 has a diameter of 0.5 inches and a length of seven inches. As will be appreciated by those skilled in the art, in some other embodiments elongated member 101 might have an alternative diameter (e.g., 0.25 inches, 0.75 inches, 1 inch, etc.), or an alternative length (e.g., 4.5 inches, 6 inches, 8.25 inches, etc.), or both.

Endcap 102 is electrically conductive and is coupled to elongated member 101, as shown in FIG. 1. In one embodiment, endcap 102 is hollow and fits snugly over an end of elongated member 101. As will be appreciated by those skilled in the art, in some other embodiments endcap 102 might be solid (i.e., having no voids), or partially hollow, rather than hollow. Similarly, in some other embodiments endcap 102 might be coupled to elongated member 101 in some alternative manner (e.g., soldered, etc.), and it will be clear to those skilled in the art, after reading this disclosure, how to make and use such alternative embodiments of endcap 102.

In one embodiment, endcap 102 comprises copper. As will be appreciated by those skilled in the art, in some other embodiments endcap 102 might comprise some other electrically-conductive material (e.g., aluminum, steel, etc.), either instead of copper, or in addition to copper.

In one embodiment, endcap 102 is cylindrical. In one such embodiment, cylindrical endcap 102 has a diameter that is 3.175 millimeters greater than the diameter of elongated member 101, and a length of 0.5 inches. As will be appreciated by those skilled in the art, in some other embodiments endcap 102 might have an alternative diameter (e.g., the same diameter as elongated member 101, a diameter that is 12.7 millimeters greater than the diameter of elongated member 101, etc.), or an alternative length (e.g., 0.75 inches, etc.), or both. Similarly, in some other embodiments endcap 102 might have a non-circular cross section (e.g., elliptical, rectangular, hexagonal, etc.), or might have a non-uniform cross-section (e.g., a cone-shaped endcap 102, etc.).

Endcap 103 is electrically conductive and is coupled to elongated member 101. In one embodiment, endcap 103 has a hole (not depicted in FIG. 1) through which wire 104 can pass, as shown in FIG. 1.

In one embodiment, endcap 103 is hollow and fits snugly over an end of elongated member 101. As will be appreciated by those skilled in the art, in some other embodiments endcap 103 might be solid (i.e., having no voids), or partially hollow, rather than hollow. Similarly, in some other embodiments endcap 103 might be coupled to elongated member 101 in some alternative manner (e.g., soldered, etc.), and it will be clear to those skilled in the art, after reading this disclosure, how to make and use such alternative embodiments of endcap 103.

In one embodiment, endcap 103 comprises copper. As will be appreciated by those skilled in the art, in some other embodiments endcap 103 might comprise some other electrically-conductive material (e.g., aluminum, steel, etc.), either instead of copper, or in addition to copper.

In one embodiment, endcap 103 is cylindrical. In one such embodiment, cylindrical endcap 103 has the same diameter as endcap 102, and the same length as endcap 102. As will be appreciated by those skilled in the art, in some other embodiments endcap 103 might have an alternative diameter (e.g., a diameter that is greater than the diameter of endcap 102, a diameter that is less than the diameter of endcap 102, etc.), or an alternative length (e.g., 0.75 inches, etc.), or both. Similarly, in some other embodiments endcap 103 might have a non-circular cross section (e.g., elliptical, rectangular, hexagonal, etc.), or might have a non-uniform cross-section (e.g., a cone-shaped endcap 103, etc.).

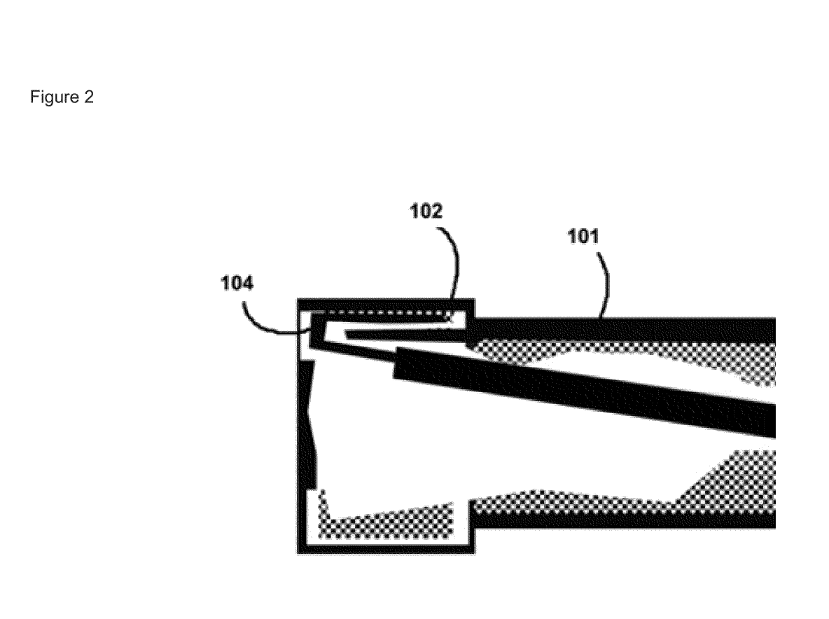

Wire 104 is electrically conductive and is coupled at one end to ground plug 105 and at the other end to at least one of elongated member 101, endcap 102, or endcap 103. In one embodiment, in which elongated member 101 is at least partially hollow, wire 104 passes through a hole in endcap 103 and through the inside of elongated member 101, as shown in FIG. 1. In one embodiment, wire 104 is coupled to both endcap 102 and to the end of elongated member 101 proximate to endcap 102. An illustrative coupling of this type is described in detail below and with respect to FIG. 2.

In one embodiment, wire 104 comprises insulated 18-gauge copper, with the insulation removed at the end coupled to at least one of elongated member 101, endcap 102, or endcap 103. As will be appreciated by those skilled in the art, in some other embodiments wire 104 might comprise some other electrically-conductive material (e.g., aluminum, steel, etc.), either instead of copper, or in addition to copper. Similarly, in some other embodiments wire 104 might comprise some other gauge of electrically-conductive material (e.g., 16-gauge wire, etc.), or might not be insulated, or both. As will further be appreciated by those skilled in the art, in some embodiments the diameters of elongated member 101 and wire 104 might be based on a desired ratio (e.g., a 10:1 ratio of the elongated member diameter to the wire diameter, etc.), or a given ratio range (e.g., between 7:1 and 13:1, at least 8:1, no more than 11:1, etc.).

FIG. 2 depicts an illustrative coupling of wire 104 to endcap 102 and elongated member 101, in accordance with one embodiment of the present disclosure in which elongated member 101 is at least partially hollow (e.g., a tube, etc.), and in which a portion of wire 104 is inside elongated member 101. As shown in FIG. 2, in accordance with this coupling, an end of wire 104 (which is uninsulated, as indicated in the figure) exits the end of elongated member 101, folds over the outside of elongated member 101, and is securely sandwiched between the inside of endcap 102 and the outside of elongated member 101.

As will be appreciated by those skilled in the art, some other embodiments might employ elements other than those depicted in FIG. 1, either instead of one or more elements depicted in FIG. 1, or in addition to elements depicted in FIG. 1, or both. For example, in some other embodiments apparatus 100 might also comprise, in addition to the elements depicted in FIG. 1, a short flexible tube (e.g., a plastic tube, a vinyl tube, etc.) extending from a hole in endcap 103, in order to prevent stressful bending of wire 104 at endcap 103.

As another example, in some other embodiments apparatus 100 might also comprise a fuse (e.g., a 0.5 A fuse, etc.) in order to prevent any potential safety issues in the case that ground plug 105 is inserted into the ground of an incorrectly-wired AC outlet. In one such embodiment, in which elongated member 101 is at least partially hollow (e.g., a tube, etc.), the fuse is situated inside elongated member 101, and apparatus 100 comprises two electrically-conductive wires, rather than a single electrically-conductive wire 104, where a first wire is coupled at one end to ground plug 105 and at the other end to a first terminal of the fuse, and where a second wire is coupled at one end to a second terminal of the fuse and at the other end to at least one of elongated member 101, endcap 102, or endcap 103.

Similarly, some other embodiments of the present disclosure might omit one or more elements depicted in FIG. 1. For example, in some other embodiments, one or both of endcap 102 and endcap 103 might be omitted. As will be appreciated by those skilled in the art, in alternative embodiments where both endcap 102 and endcap 103 are omitted, wire 104 might be coupled to a sealed end of elongated member 101 (e.g., via a soldered connection, etc.), or to some other location of elongated member 101.

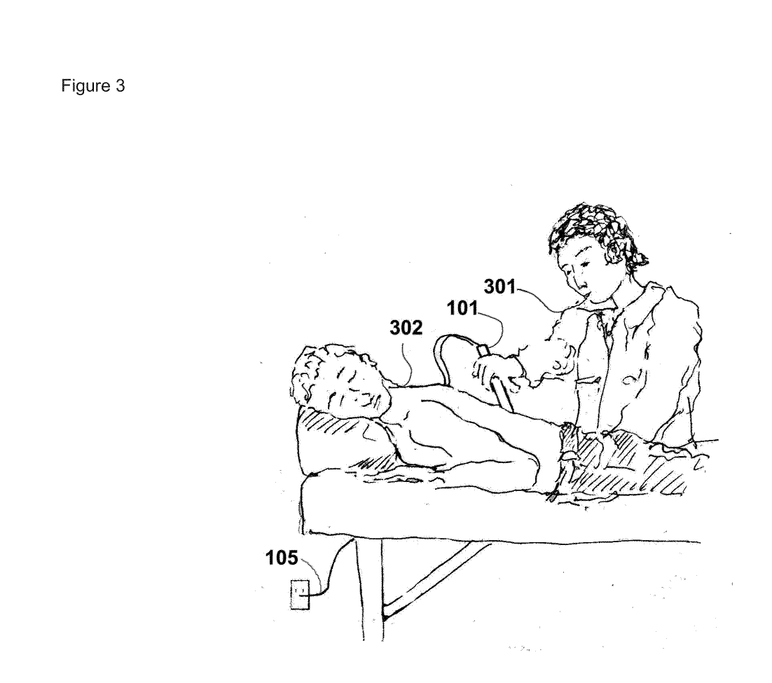

FIG. 3 depicts an illustrative use of apparatus 100, in accordance with one embodiment of the present disclosure. As shown in FIG. 3, ground plug 105 of apparatus 100 has been inserted into a ground receptacle of an alternating-current outlet, and while ground plug 105 is in the ground receptacle, a therapist 301 applies pressure via elongated member 101 to one or more acupressure points of a patient 302. After administration of acupressure has been completed, ground plug 105 can then be removed from the ground receptacle. As will be appreciated by those skilled in the art, embodiments of the present disclosure can also be used for self-administration (e.g., administration by patient 302 on him-or-herself, etc.).

As will further be appreciated by those skilled in the art, embodiments of the present disclosure can also be used independent of acupressure therapy, to drain excess charge from a person over a period of time, and thereby provide the various health benefits of such draining. For example, in one embodiment, while ground plug 105 is connected to a ground receptacle of an alternating-current outlet, elongated member 101 can be conveniently held by a person, or can be conveniently secured to a person by inserting elongated member 101 between the person's waist and a belt worn by the person.

It is to be understood that those skilled in the art can easily devise variations of embodiments of the present disclosure, and that scope is to be determined by the following claims.

What is claimed is:

1. An apparatus for administering acupressure, the apparatus comprising:
an electrically-conductive elongated member to be applied to one or more acupressure points of a person; and
an electrically-conductive ground plug, coupled via an electrically-conductive wire to the elongated member, to be inserted into a ground receptacle of an alternating-current outlet during application of the elongated member to the one or more acupressure points;
wherein the ground plug and wire enable charge drainage from the person during application of the elongated member to the one or more acupressure points.

2. The apparatus of claim 1 wherein the elongated member is a tube.

3. The apparatus of claim 1 wherein the elongated member is cylindrical.

4. The apparatus of claim 1 wherein the elongated member has no voids.

5. The apparatus of claim 1 further comprising a first endcap that is coupled to one end of the elongated member, and a second endcap that is coupled to the other end of the elongated member, wherein the first endcap has a hole though which the wire can pass and contact at least one of the elongated member, the first endcap, or the second endcap.

6. The apparatus of claim 1 wherein the diameter of the elongated member is at least 10 times as large as the diameter of the wire.

7. The apparatus of claim 1 wherein the length of the elongated member is between 5 and 7 inches inclusive.

8. A method of administering acupressure, the method comprising:
inserting into a ground receptacle of an alternating-current outlet an electrically-conductive ground plug that is coupled via an electrically-conductive wire to an electrically-conductive elongated member; and
applying the electrically-conductive elongated member to one or more acupressure points of a person while the ground plug is in the ground receptacle of the alternating-current outlet;
wherein the ground plug and wire enable charge drainage from the person during application of the elongated member to the one or more acupressure points.

9. The method of claim 8 wherein the elongated member is a tube.

10. The method of claim 8 wherein the elongated member is cylindrical.

11. The method of claim 8 wherein the elongated member has no voids.

12. The method of claim 8 wherein the diameter of the elongated member is between 0.25 and 1.0 inches inclusive.

13. The method of claim 8 wherein the length of the elongated member is between 5 and 7 inches inclusive.

14. An apparatus for administering acupressure, the apparatus comprising:
an electrically-conductive elongated member to be applied to one or more acupressure points of a person, wherein the elongated member is at least partially hollow;
an electrically-conductive ground plug;
a fuse located inside the elongated member;
a first electrically-conductive wire coupled at one end to the ground plug and coupled at the other end to a first terminal of the fuse; and
a second electrically-conductive wire coupled at one end to a second terminal of the fuse, and coupled at the other end to at least one of the elongated member or an electrically-conductive object coupled to the elongated member;
wherein the ground plug is to be inserted into a ground receptacle of an alternating-current outlet during application of the elongated member to the one or more acupressure points; and
wherein the ground plug, the first wire, and the second wire enable charge drainage from the person during application of the elongated member to the one or more acupressure points.

15. The apparatus of claim 14 wherein the elongated member is cylindrical.

16. The apparatus of claim 14 wherein the object is a first endcap coupled to an end of the elongated member.

17. The apparatus of claim 16 wherein the second electrically-conductive wire folds over the outside of the elongated member and is sandwiched between the inside of the first endcap and the outside of the elongated member.

18. The apparatus of claim 16 further comprising a second endcap coupled to the other end of the elongated member, wherein the second endcap has a hole though which the first wire can pass.

19. The apparatus of claim 14 wherein the diameter of the elongated member is between 0.25 and 1.0 inches inclusive.

20. The apparatus of claim 14 wherein the length of the elongated member is between 5 and 7 inches inclusive.
